(19) United States
(12) Patent Application Publication
(10) Pub. No.: US 2007/0004022 A1
(43) Pub. Date: Jan. 4, 2007

(54) Industrial fermenting production process of Hirsutella hepiali Chen & Shen of anamorphic fungi related to Chinese Cordyceps Sinensis

(76) Inventor: Nanying Shen, Hangzhou (CN)

Correspondence Address: HAMRE, SCHUMANN, MUELLER & LARSON, P.C., P.O. BOX 2902, MINNEAPOLIS, MN, 55402-0902, US

(21) Appl. No.: 11/450,747

(22) Filed: Jun. 9, 2006

Related U.S. Application Data
(63) Continuation-in-part of application No. PCT/CN05/00565, filed on Apr. 25, 2005.

(30) Foreign Application Priority Data
May 31, 2004 (CN) 200410037116.9

(51) Int. Cl. A01G 1/04; C12N 1/14

(52) U.S. Cl. 435/254.1; 47/1.1

(57) ABSTRACT

The present invention discloses a fermenting production process of Hirsutella hepiali Chen & Shen for industrial purpose. It contains steps: a. Isolating new stain from original source; b. identifying whether the stain can grow stroma or not; c. culturing the strain in solid medium for rejuvenescence purpose; d. Secondly culturing the strain in liquid culture medium; e. fermenting. Said method provides a fermenting production process, which can continually identify whether the anamorphic fungi related to Chinese Cordyceps sinensis change or not, whether it retains the property of original strain or not. It also can be modified continually. According to these processes, the quality of the obtained product will be stable and the property will be retained stable for quite a long time. Therefore this method overcome the problems exist in the art that change of the strain, instability of quality of the product and so on.

### CROSS REFERENCE TO RELATED APPLICATIONS

This application is a continuation-in-part of, and claims priority to, co-pending Patent Cooperation Treaty application PCT/CN2005/000565, filed Apr. 25, 2005, and entitled “Industrial Fermenting Production Process of Hirsutella hepiali Chen & Shen of Anamorphic Fungi Related to Chinese Cordyceps Sinensis,” which claims priority to Chinese application Serial No. 200410037116.9, filed May 31, 2004, which is herewith incorporated by reference in its entirety.

### TECHNICAL FIELD

The invention relates to the producing method of an asexual variety of Chinese caterpillar fungus (Cordyceps sinensis (Berk) Sacc), whose scientific name is Hirsutella sinensis Liu, Guo, Yu & Zeng, which has a synonym of Hirsutella hepiali Chen & Shen. Particularly the invention relates to an industrial fermentation-producing method for the asexual variety of the Chinese caterpillar fungus (Hirsutella sinensis Liu, Guo, Yu & Zeng).

### BACKGROUND OF THE INVENTION

The asexual Chinese caterpillar fungus is distributed in Qinghai-Tibet Plateau, China, mainly growing in high-altitude areas, 4500-5200 meters above the sea level, and it has very special demands on the ecological conditions, i.e., not only the temperature and humidity should be very low and it should be frozen in the ice for 8 months in a year, but also it has very strict demands on the light quality and air composition. However, it has a very high medicinal value. Particularly it has a two-way regulating action to the human immunologic function and internal secretion, and has an anti-rejection action on organ replantation. Over recent years, it has been discovered during researches on kidney replantation that the drugs with the Chinese caterpillar fungus as the active pharmaceutical ingredient have a very good detoxification action against the toxic side effects caused by the long-term administration of the anti-rejection drug cyclosporine (Cs) A and its accompanied drugs.

Fungi are lower plants. If they grow under untoward environment conditions on a long-term basis, they will gradually change their original species properties, and therefore the power of this drug's excellent effectiveness will be gradually reduced over time to zero. The change in the original fungal properties can be attributed to causes of two aspects: No. 1: the change of species: because the properties of fungi of various species that grow under the same or similar ecological conditions during the asexual period, i.e. the hyphal period, are very similar, and on the living tissue on which the Chinese caterpillar fungus grows there are also dozen of fungi of various other species, some of which can be temporarily in an inhibited state due to sterilization and will wake up later and rapidly grow to replace the Chinese caterpillar fungus. No. 2: Even if the “species” does not change, as it originally grows at a high-altitude area with very low temperature and a 8-month freezing period in the year, but in the artificial culture it is often cultivated at 16-18° C., and a culture medium that can make the hyphae grow fast is used in order to make the Chinese caterpillar fungus grow fast and yield high output; moreover the light quality and air components are also greatly different from its original conditions, hence it reproduces from generation to generation under untoward conditions, and consequently the fugal strain that grows fast will be selected in the process and the inherent properties of the Chinese caterpillar fungus gradually change and its original effective ingredients will gradually diminish.

After the hyphae have matured in a fermentation vessel, the hyphae are extracted generally via the filtration methods with the spun yarn bags in a centrifuge. Because the sizes of the spun-yarn bag pores are not even, the high-speed rotation of the centrifuge will cause loss of some hyphae. Moreover, because of the centrifugal pressure, the filtered hyphae are stuck to the filtration bags, forming a solid layer containing hyphae, which must be torn into flakes with hand. The manual operation will result in the thickness unevenness of the flakes containing hyphae and accordingly cause the inconsistency of heating and oxidation while those fakes are heated, and the color and ingredients of the product will be affected. Furthermore, the manual operation increases the chances of the product's being infected by bacteria. Therefore on the basis of practice and test of many years, our talented technical professionals have adopted the hypha-extracting process of vacuum drum filtration and subsequently tunnel drying instead and as a result have upgraded the output and quality of the hyphae.

Because the Chinese caterpillar fungus has a very high medicinal value, since the 80s of the last century about 20 to 30 institutes over China have conducted research on this fungus. In order to obtain the fungal strain, a laboratory should be established in a growing area. Once a laboratory was established in a growing area that was located at the altitude of 3700 meters above the sea level and it had the highest temperature of 16° C. in the summer. Moreover a testing base was established in a growing area of the Chinese caterpillar fungus with the altitude of 4,600 meters above the sea level, where mature sexual spores (ascus spores) of the Chinese caterpillar fungus could be obtained. These spores were introduced into an Erlenmeyer flask, and then they completed their developmental cycle in at the sterile conditions, where they grew from single wild ascus spores (or living tissue blocks) to fungal colonies, then to conidia, subsequently over fruiting bodies (correspond to the tassels of some higher plants) to ascus spores that were completely the same as wild ascus spores and correspond to the seeds of higher plants.

Moreover, as for culture media, the artificially prepared culture media cannot meet the need of the Chinese caterpillar fungus, because it is a very selective insect-parasitizing fungus with very strict demands on the host, which is specifically the caterpillar of the bat moth caterpillar (Thitarodes armoricanus), and it has very strict special demands on the nutrition. The caterpillar of bat moth caterpillar lives underground in the high altitude mountain areas and its distribution regularity has been studied. The caterpillars to be parasitized were used as the culture medium in the primary isolation of the fungal strains. After the acquirement of the fungal strains, various fungal culture media were then tested to select the components the fungus preferred. Through repeated combinations, an optimum culture medium have been finally prepared, and in it the fungus could complete its developmental cycle in an Erlenmeyer flask at the sterile conditions

Furthermore, to obtain the fungal strains through isolating the living tissues is also very important. If the fungal strains are collected after their stromata have grown above the ground, the isolation of the fungus will become more difficult because many other fungi have been mixed into the caterpillar tissue. When conducting research on the high-altitude area, we often collected the host caterpillars for use as cultural media. Every year during the period from last 10 days of August to the first 10 days of September when the surface layer of the local area begins to be frozen up, often about 10 hibernating caterpillars, which have just been parasitized, can be dug up at the same time, and only stromata the size of a millet grains on the top of the caterpillar head can be seen and it is also called “bud”. These parasitized hibernating caterpillars live through the winter at a frozen state in ice for that year, and in the May of the next year after the ground is thawed, the stromata will emerge above the ground. To isolate the Chinese caterpillar fungus before winter by using the living tissue of the hibernating caterpillar is most likely to be successful, because before winter the tissue of the hibernating caterpillar are very hard and clean and the Chinese caterpillar fungus is also at its prime life stage.

After a Chinese caterpillar fungus is collected, to identify the true species is critically important. To do research in the growing area, it is convenient to establish a testing base where the sexual spores of the Chinese caterpillar fungus can grow to the mature stage, in the same area the fungus is collected. This kind of areas is generally very far away from inhabitants and is at a high altitude (above 4600 meters). It is important to remove the Chinese caterpillar fungi that grow naturally, keeping a portion of their own soil intact, and replant them together in a new location prior to the introduction of cattle and sheep for grazing in the summer. It would be good to isolate that location by adding barbed wire. In addition, guards, who would live there for that time period, should be employed temporarily to protect the fungi from damage by the cattle and sheep during the summer months. A large number of mature ascus spores can be collected by covering the fungi with sterile paper bags before the ascus spores mature and spurt out. The reason for doing this is that only the ascus spores that grown and mature at the original place of the fungi can sprout. Because the spores of the Chinese caterpillar fungus are relatively big and their surfaces are inevitably infected by bacteria and much smaller fungal spores, it is impossible to conduct sterilization with routine sterilizing methods, because this way the fungi themselves will be killed. With a large number of mature ascus spores, the spores can be rinsed out of the sterile paper bags with the boiled soil lixiviation (soil:water=1:1) of the growing area to prepare the sucrose solutions with the respective concentration of 20%, 40%, 60% and 80% and then centrifugalize the solution of each concentration at a high speed for 30 min. The purpose is to eliminate fungi of other species and spores of them, which have various specific gravities, from the surfaces of ascus spores, and therefore make the ascus spores of the Chinese caterpillar fungus relatively pure and clean, to achieve the effect of sterilization. Then a dilute spore solution is prepared in a sterile soil solution and then is spread on a thin-layer culture medium at the bottom a small evaporating dish. After that the dish is let to stand at 15-20° C. for 3-6 days until the ascus spores germinate. The ascus spores, around which there are no fungi of other species growing, are identified under a microscope, and then carefully removed with an inoculating needle and replanted in a sterile small evaporating dish. Each single ascus spore is followed and observed under the microscope in its growing process of hyphae over colonies to conidia. If the conidia are the same as the ones that grow from the fungus isolated from the living tissue of the bat moth caterpillar, they are proved as those of Chinese caterpillar fungus. Then they are transferred to an Erlenmeyer flask of above 500 ml and cultivated at the low temperature of below 10° C. and as a result some of them can form stromata. Moreover, via the treatment of black light lamp and ultraviolet lamp, individual stromata can form ascocarps that will spurt ascus spores. The ascus spores are identical to the wild ones and hence the sexual spores are also identified as identical to the relevant wild ones. However the testing process cost a very long time and is very difficult. In order to simplify the process, the identification can be realized via the comparison of the conidia, which have grown from each ascus spore in dishes or the slant cultures of test tubes, with the conidia that have grown from the fungal strain, which is isolated from the living tissue and also has developed stromata as well as conidia, in the characters of size, shape, color and conidiophores. Therefore the true fungal species can be identified through the conformity of these characters. Thus, the identifying process runs through the whole developmental cycle and provides proofs that have been acknowledged by the biological circle.

This is described in a patent: Chinese caterpillar fungus and the method of its artificial cultivation (Chinese Patent: No. 85101971.4).

In order to achieve sufficient products and put them into the pharmaceutical market, a bioengineering fermentation approach must be adopted. Because of the special demands of this fungus, to complete the relevant industrial fermentation is very difficult. The fermentation cultivation of common fungi can be completed at above 25° C. in about 10 days. However, the Chinese caterpillar fungus must be cultivated at below 20° C. for about 40 days via a 4-step fermentation process. During cultivation of so long a time, it is very difficult to avoid contamination. As described in a patent: fermentation-producing method of the Chinese caterpillar fungus (Chinese Patent: No. 97110448.4), a method has been used to prevent the contamination with the characters of putting the fungal strains into the step-1 starter vat and fermenting it at below 20° C. for 6-8 days. Then the culture medium containing the fungal strain is expanded by 10 times and is fermented stepwise until the needed amount is obtained. Subsequently the culture is removed from the can and filtered. The composition of liquid culture medium: Carbon source of 0.5-5%, nitrogen source of 0.2-5%, trace elements, a little amount of vitamins and water. Via this approach the industrial production of a large scale for the Chinese caterpillar fungus has been realized with low production cost and high speed. Nevertheless, with the continuous production and the reproduction of the fungus from generation to generation, the product has been continuously undergoing variation. Hence the product related to this patent still has shortfalls in the aspects of keeping the true fungal species and the inherent fungal properties.

### SUMMARY OF THE INVENTION

The first purpose of the invention is to overcome said shortfalls and create a production approach via which the species of the Chinese caterpillar fungus can be continuously tested to make sure that it still maintains the original species, and modifications of some process can be done to keep the inherent fungal properties. As a result the stability of product quality can be guaranteed and the pharmaceutical effectiveness of the product can be stabilized on a long-term basis.

The second purpose of the invention is to create an improved hypha extracting and drying method that can upgrade output and improve product quality.

The above technological problems mentioned above have been solved via the following technical plans.

An industrial fermentation-producing method for the asexual Chinese caterpillar fungus comprises the following steps:

1. Isolation of new fungal strains: The new fungal strains are isolated in the growing area and introduced into Erlenmeyer flasks for cultivation at 10-20° C.;

2. Test: The newly obtained new fungal strains are cultivated at 0-10° C. to test whether the new strains can develop stromata. The fungal strains that can develop stromata will be used in production;

3. Rejuvenating cultivation: The fungal strains, which have been used in the production and have been purified and reproduced for more than 10 generations, are rejuvenated via cultivation. They are reproduced at the low temperature of 0-10° C. for more than 5 generations. The conidia and conidiophores that have grown via cultivation should be identical to those of the fungi that have been isolated from the growing area and can develop stromata. This should be proved via comparison. The culture medium used in said step should be a solid culture medium, with the formulation (calculated as 1000 g); Beef tea (1:2) 300-500 g, lactalbumin hydrolysate 5-10 g, yeast powder 10 g, glucose 20-40 g, milk 100-200 g, nucleic acid 0.5-1 g, magnesium sulfate 0.1 g-0.4 g, potassium dihydrogen phosphate 0.6-1 g, compound vitamin 0.5-2 g, agar powder 15-20 g, water 300-600 g. The pH value of said medium is 7-8;

4. The second cultivation: The fungal strains that meet relevant demands are introduced into a liquid culture with the temperature of 12-20° C. for cultivation, said culture is put on a rocking device and cultivated for 6-12 days;

5. Fermentation: Said cultivated fungal strains are put into a step-1 starter vat and fermented at 12-20° C. in a liquid culture medium for 8-12 days. The culture medium is expanded by 8-12 times, fermented stepwise, then removed from the vat and dried. Said liquid culture medium (calculated from weight percent) comprises any one or more of the flowing materials: carbon source 0.5-5%, nitrogen source 0.5-2%, trace elements 0.1-0.2%, and f vitamins 0.1-0.2%; the remaining part is water.

### BRIEF DESCRIPTION OF THE DRAWINGS

FIG. 1 shows a graph of the fungal growth during fermentation for the invention.

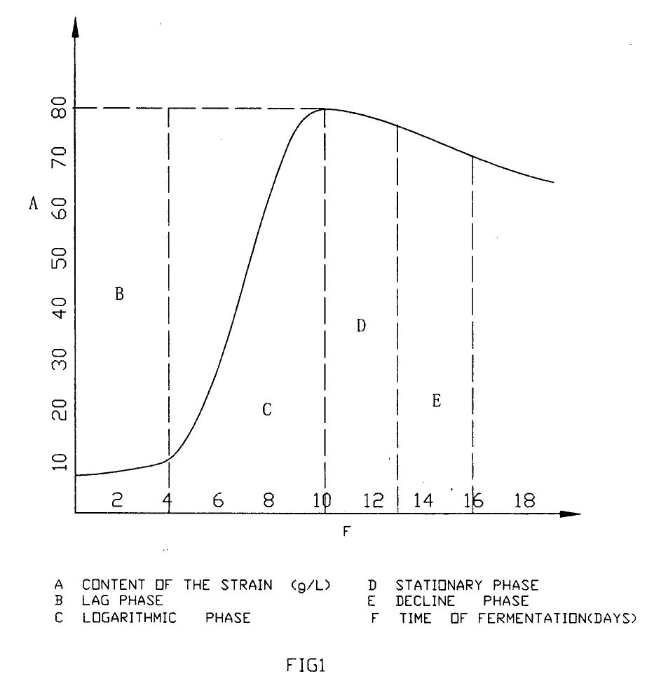

FIG. 2 shows a picture of the fungal strain while it is growing “stromata.”

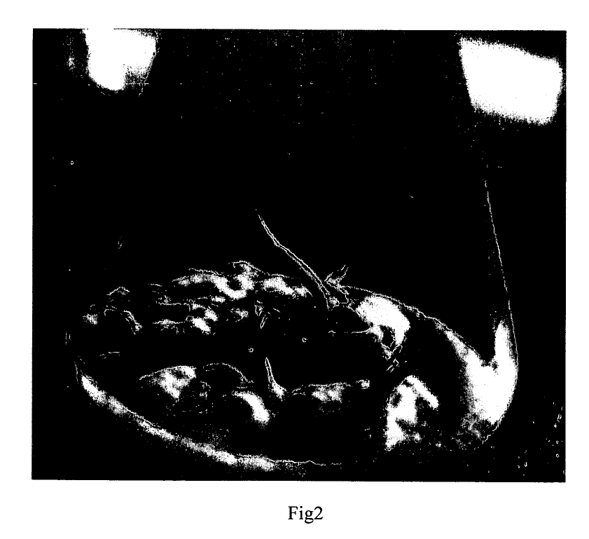

FIG. 3 shows the therapeutic effects on the kidney replantation in Working Group 1—comparing Working Group 1 and Natural Chinese Caterpillar Fungus Group.

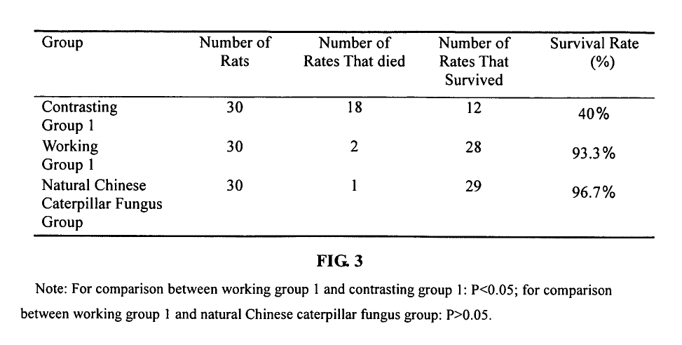

### DETAILED DESCRIPTION

The first purpose of the invention is to overcome said shortfalls and create a production approach via which the species of the Chinese caterpillar fungus can be continuously tested to make sure that it still maintains the original species, and modifications of some process can be done to keep the inherent fungal properties. As a result the stability of product quality can be guaranteed and the pharmaceutical effectiveness of the product can be stabilized on a long-term basis.

The second purpose of the invention is to create an improved hypha extracting and drying method that can upgrade output and improve product quality.

The above technological problems mentioned above have been solved via the following technical plans.

An industrial fermentation-producing method for the asexual Chinese caterpillar fungus comprises the following steps:

1. Isolation of new fungal strains: The new fungal strains are isolated in the growing area and introduced into Erlenmeyer flasks for cultivation at 10-20° C.;

2. Test: The newly obtained new fungal strains are cultivated at 0-10° C. to test whether the new strains can develop stromata. The fungal strains that can develop stromata will be used in production;

3. Rejuvenating cultivation: The fungal strains, which have been used in the production and have been purified and reproduced for more than 10 generations, are rejuvenated via cultivation. They are reproduced at the low temperature of 0-10° C. for more than 5 generations. The conidia and conidiophores that have grown via cultivation should be identical to those of the fungi that have been isolated from the growing area and can develop stromata. This should be proved via comparison. The culture medium used in said step should be a solid culture medium, with the formulation (calculated as 1000 g); Beef tea (1:2) 300-500 g, lactalbumin hydrolysate 5-10 g, yeast powder 10 g, glucose 20-40 g, milk 100-200 g, nucleic acid 0.5-1 g, magnesium sulfate 0.1 g-0.4 g, potassium dihydrogen phosphate 0.6-1 g, compound vitamin 0.5-2 g, agar powder 15-20 g, water 300-600 g. The pH value of said medium is 7-8;

4. The second cultivation: The fungal strains that meet relevant demands are introduced into a liquid culture with the temperature of 12-20° C. for cultivation, said culture is put on a rocking device and cultivated for 6-12 days;

5. Fermentation: Said cultivated fungal strains are put into a step-1 starter vat and fermented at 12-20° C. in a liquid culture medium for 8-12 days. The culture medium is expanded by 8-12 times, fermented stepwise, then removed from the vat and dried. Said liquid culture medium (calculated from weight percent) comprises any one or more of the flowing materials: carbon source 0.5-5%, nitrogen source 0.5-2%, trace elements 0.1-0.2%, and f vitamins 0.1-0.2%; the remaining part is water.

As the culture medium is the nutritional material that supports the growth, reproduction, catabolism and anabolism, the appropriate composition and formulation of the culture medium plays a significant role in the growth, development, fermentation unit, extracting process and the quality and output of the final product. An optimum formulation of the culture medium can bring the biological synthetic ability of the productive fungal strain into full play, so that the maximal productive effect can be reached and the fermentation efficiency can be upgraded. Therefore, importance must be attached to the composition of the culture medium. The determination of an excellent fermentation culture medium usually need a long-term test of productive practice and should be improved continuously.

As one of the components of the culture medium, the carbon source can not only act as an energy source, but also as the cell material for the fungal strain. During the growing process of the microorganism, the carbon source mainly meets the needs of the fungal growth, the consumption for maintaining the fungal life, and the consumption of the accumulated metabolic products. The phenomenon can be expressed with the formula: 1/Y_{X/S}=m/μ+1/Y_{G}, in which m symbolizes the carbon source, and 1/YG symbolizes the ratio of the substrate consumption, which occurs during the fungal growth or fermentation process, to the mass of fungal tissue produced. From the formula, it can be seen the carbon source is the indispensable and the most important substrate during the growth and metabolism of the fungal strain, and the fungal strain should keep a carbon balance during the growing process. The inventor has discover on the basis of a long-term research that the formulation of the solid culture medium (calculated as 1000 g) should be as follows: beef tea (1:2) 300-500 g, lactalbumin hydrolysate 5-10 g, yeast powder 10 g, glucose 20-40 g, milk 100-200 g, nucleic acid 0.5-1 g, magnesium sulfate 0.1 g-0.4 g, potassium dihydrogen phosphate 0.6-1 g, compound vitamin 0.5-2 g, agar powder 15-20 g, and water 300-600 g; the liquid culture medium comprises any one or more of the following materials: carbon source 0.5-5%, nitrogen source 0.5-2%, trace elements 0.1-0.2%, and vitamins 0.1-0.2%; the remaining part is water. Therefore not only the carbon balance in the asexual Chinese caterpillar fungus (Hirsutella hepiali Chen & Shen) can be maintained, but also the maximal speed of fugal fermentation can be obtained. As a result, the yield of the fermenting product is greatly elevated. In the growing process of the fungal strain, ATP alone is far from being sufficient as it only acts as the energy source, and other materials are needed for synthesizing the cell, such as the nitrogen source, trace elements, vitamins etc., which usually enter the cell with the original valence. In the growing process of the fungal strain, there may appear two situations: one is that there exist plenty of materials for synthesizing the fungal cell, but the ATP that results from decomposing the carbon source becomes the limiting factor, and the other is that there is an excessive amount of ATP, but the materials for synthesizing the fungal cell becomes the limiting factor. On the basis of a long-term research, the inventor has successfully designed a formulation of the solid culture medium and that of the liquid culture medium, which have solved the problem of the mutual limitation of the ATP and fungal cell materials, brought their applied effects into full play and therefore upgraded the fermentation yield of the asexual Chinese caterpillar fungus. The composition of the formulation (calculated as 1000 g) is as follows: beef tea (1:2) 300-500 g, lactalbumin hydrolysate 5-10 g, yeast powder 10 g, glucose 20-40 g, milk 100-200 g, nucleic acid 0.5-1 g, magnesium sulfate 0.1 g-0.4 g, potassium dihydrogen phosphate 0.6-1 g, compound vitamin 0.5-2 g, agar powder 15-20 g, and water 300-600 g; the liquid culture medium comprises any one or more of the following materials: carbon source 0.5-5%, nitrogen source 0.5-2%, trace elements 0.1-0.2%, and vitamins 0.1-0.2%; the remaining part is water. Among the physical factors that affect the growth and fermentation of the fungal body, the temperature play the most important role as the heat not only affects the surface of the fungal body, but also will be transmitted into the fungal body and affect the all the internal structural materials of the fungal body. Because the asexual Chinese caterpillar fungus has very high demands on the temperature and the fungal strain itself also produces heat during fermentation, if the temperature is not controlled well the fungi will die in a great scale. This invention controls the temperature within the range of 12-20° C., which is the optimum temperature condition for the fungal strains. The growth of the fugal body needs a certain acidity or alkalinity (expressed as pH value), which will play a very important role in the growth and the metabolic product formation of microorganisms, and different fungi need respectively different pH value. Moreover, even the fungus of the same species may produce different fermentation products because of relevantly different pH values, and the optimum pH value for fungal growth often does not conform to the optimum pH value for fermentation, which is mainly due to the following reasons: 1) the pH value change of the fermentation liquid results in the electric change in the cellular plasma membrane; 2) the pH value in the fermentation liquid directly affects the activity of the enzymes and different enzymes need respectively different pH value to produce the maximal activity; 3) the pH value of the fermentation liquid affects the dissolution of some important nutritional materials and mesostates in the culture medium and therefore affects the utilization of the these materials by the fungal strain. In the invention, the pH value of the solid culture medium is adjusted to 7-8 and that of the liquid culture medium is adjusted to 7.0-7.5. These are the optimum pH range value ranges of the fungus and can bring about the maximal growth speed of the fungal strain.

Preferably, the formulation of the solid culture medium (calculated as 1000 g) mentioned above should be: beef tea (1:2) 300-500 g, lactalbumin hydrolysate 5-10 g, yeast powder 1 g, glucose 20-40 g, milk 100-200 g, nucleic acid 0.5 g, magnesium sulfate 0.2 g, potassium dihydrogen phosphate 1 g, compound vitamin 1 g, agar powder 15 g, and water 400-600 g. The pH value of the culture medium should be 7.2-7.6.

Preferably, in the liquid culture medium, the nitrogen source comprises any one or more of the following materials: silkworm chrysalis powder, protein peptone, milk powder, yeast powder and lactalbumin hydrolysate, and their contents (calculated from weight percent) are 0.5-2%; the carbon source comprises any one or more of the following materials: royal jelly, oat powder, wheat gluten, saccharose, corn flour and glucose, and their contents (calculated from weight percent) are 0.1-0.2%. Said materials in the medium culture are inexpensive and readily available, and therefore they can result in very good cultivation effects.

Preferably, in the liquid culture medium for every 1000 g water, there are silkworm chrysalis powder 15 g, protein peptone 2 g, corn flour 10 g, wheat gluten 15 g, glucose 20 g, magnesium sulfate 0.3 g, dipotassium hydrogen phosphate 0.6 g, and the pH value of said liquid culture medium is 7.0-7.5.

Preferably, in the liquid culture medium for every 1000 g water, there are royal jelly 2 g, oat powder 20 g, milk powder 15 g, saccharose 20 g, magnesium sulfate 0.3 g, dipotassium hydrogen phosphate 0.6 g, vitamin 1 g, and the pH value of said liquid culture medium is 7.0-7.5.

Preferably, said compound vitamin is prepared from bleeding vitamin B1, vitamin B2, thiamin and riboflavin with water.

Another character of the invention is that after said fermentation step is finished, the liquid culture medium is introduced into a high-level storing vessel and slowly transferred into a drum vacuum dryer. As a result the hyphae are evenly adsorbed at the bottom and then gradually sent through a 15-20 m long drying tunnel. When the culture medium containing the hyphae reaches the outlet, it has already lost 70-80% water. Subsequently it is cut into flakes of approx. 1.0×1.0 cm with a cutter. Finally these flakes are boiled, dried, pulverized and sifted to obtain the finished products. When the culture medium containing the hyphae reaches the outlet, it has already lost 70-80% water. Subsequently it is cut into flakes of approx. 1.0×1.0 cm that contain hyphae. Finally these flakes are quickly dried in a boiling dryer, pulverized and sifted to obtain the finished products.

The fungal powder can also be obtained by the following process: Homogenizing the artificially fermented culture medium, filtering it through a membrane, and drying it via spray-drying. By using the spray-drying method, the quantity of fungal derivants in the culture medium is increased, and therefore the effectiveness is higher with the ingredients being more similar to those of natural Chinese caterpillar fungus.

Hence, the advantages and the effects of the invention are as follows: firs of all, as we have gone to the growing area to collect the new fungi every year, and checked the true identity of the fungal strain by rejuvenating the fungi used, completing the whole cycle of the growth and development during the cultivation period while observing whether the fungi can develop stromata, and appraised the existence of the inherent genetic properties on the basis of the characters of their asexual spores, therefore we can continuously ensure the true identity of the asexual Chinese caterpillar fungus (Hirsutella hepiali Chen & Shen), maintain its unique properties and keep the stability of the product quality as well as its stable medicinal effects; moreover, because of the adoption of the culture medium formulation and the physical conditions of the invention, it has been tested and proved via practice that the product produced by using the methods of the invention maintains the effectiveness that is basically identical to the effectiveness of the natural Chinese caterpillar fungus in treating various deficiency syndromes, and the anti-rejection effect used after organ replantation (especially after the kidney replantation) and detoxification effect against Cs and other anti-rejection drugs that are identical to the effects of the natural Chinese caterpillar fungus. On the other hand, the price of the product is far lower than that of the natural Chinese caterpillar fungus and therefore has a wide market prospect; finally because the invention adopts the drum vacuum drying method for extracting and drying the product, the yield of the hyphae has been upgraded by 5-20% and the quality has been improved greatly. Its color has been changed from the original gray and deep brown to the present brownish yellow or deep brown that conforms to the color of the surface of the natural Chinese caterpillar fungus, and shows the uniformity of the oxidization. Consequently the quality of the product is very close to that of the natural Chinese caterpillar fungus.

### WORKING EXAMPLES

The present invention now will be described in further detail through the following working examples, but the present invention is not limited to or by these examples.

### Working Example 1

We have isolated new fungal strains in the growing area every year and then cultivated them at 10° C. through the sexual and asexual reproductive portions of their developmental cycle, during which stromata and conidia develop. Then the obtained fungal strains are introduced into 500 ml Erlenmeyer flasks for cultivation at 0-10° C., in order to identify whether the cultivated fungal strains are true Chinese caterpillar fungi. I.e., if any one fungal strain can develop stromata, it can be identified as a true Chinese caterpillar fungus, and if it can not develop stromata, it should be eliminated. The fungal strains, which have been utilized in production and have been purified and reproduced for more than 10 generation, must be rejuvenated in a high-nutritional solid culture medium at 0-10° C. for more than 5 generations. If as a result of comparison the conidia and conidiophores grown from rejuvenated fungal strains are identical to those from the fungal strains that are newly isolated from the growing area and can develop stromata in colonies in the Erlenmeyer flasks, it has been proved that there occurs no variation or very little variation, so the fugal strains can be still utilized in production

The formulation of the culture medium for the above-mentioned cultivation (calculated as 1000 g) comprises beef tea 300 g, lactalbumin hydrolysate 10 g, yeast powder 1 g, glucose 40 g, milk 200 g, nucleic acid 0.5 g, magnesium sulfate 0.1 g, sodium dihydrogen phosphate 1 g, compound vitamin 2 g, agar powder 15 g and water 300 g; the pH value of the culture medium is 7.

The fungal strains cultivated via said procedure are put into the culture medium that is put on a rocking device with the temperature of 12° C., and then are introduced into a step 1 starter vat for fermentation at 12° C. for 12 days. After that the culture is expanded by 12 times and fermented stepwise. Until the culture medium is put into the production vessel of 20 to 30 tons at step 4, the fermentation is conducted in the liquid culture medium. The liquid culture medium (calculated from weight percent) comprises silkworm chrysalis powder 15 g, protein peptone 2 g, corn flour 10 g, wheat gluten 15 g, glucose 20 g, and magnesium sulfate 0.3 g, dipotassium hydrogen phosphate 0.6 g in every 1000 g water; the pH value of said liquid culture medium is 7.0.

After that, the liquid culture medium (containing hyphae) is introduced into a drum vacuum dryer, and thereupon the hyphae are adsorbed at the bottom of the filter and slowly transferred via a belt conveyor through a 15 m long constant-temperature drying tunnel at 60° C. while the hyphae are heated uniformly and oxidized consistently. When the culture medium containing the hyphae reaches the outlet, it has already lost 80% water. Subsequently it is cut into flakes of approx. 1.0×1.0 cm. Finally these flakes are quickly dried in a boiling dryer, pulverized and sifted to obtain the finished products.

Contrasting Example 1: The fungal strains are put into a liquid culture medium that is put on a rocking device, and cultivated at 12° C. for 12 days. After that the culture is transferred into a step 1 starter vat and fermented at 12° C. for 10 days. Then the volume of the culture medium is expanded by 10 times and fermented stepwise until the needed quantity is reached. Subsequently the culture medium is removed from the vat and filtered to obtain the finished product. Said liquid culture medium (calculated from weight percent) comprises silkworm chrysalis powder 1.5%, protein peptone 0.1%, corn flour 2%, saccharose 2%, and a little dipotassium hydrogen phosphate and magnesium sulfate; the remaining part is water.

### Testing Example 1

The preparation of the Chinese caterpillar fungus extract: The artificial Chinese caterpillar fungus powder and the beforehand-prepared natural Chinese caterpillar fungus extractive were extracted with alcohol or via enzymatic hydrolysis again. The obtained extracts were blended and contracted, added with 3% polysorbate and stirred. The concentration of the fungus powder in the obtained extract was 0.8 g/g.

The preparation of a renal intoxication model: A renal intoxication model of the white rats was prepared with the hypodermic injection of Cs A (25 mg/kg).

Testing Method:

The renal intoxication model was prepared with ninety healthy rats with the weights of 350 g+/−50 g, which were randomly selected and divided into 3 groups of 30 rats. The product of the invention was orally administered as an extract to one of the groups (working group 1), and jinshuibao (a similar product available on the market used for contrasting) and the natural Chinese caterpillar fungus were respectively orally administered to the other two groups (contrasting group 1 and natural caterpillar fungus group) as extracts on the basis of above-mentioned dosage, twice daily. These groups were observed daily for 100 days and their survival rates were determined.

The result shows that in the aspect of oral administration for treating the toxic effect caused by Cs A, the therapeutic effect of the fermented product of the invention from asexual Chinese caterpillar fungus on rats in the working group 1 is significantly higher than that of jinshuibao on those in the contrasting group 1, and the survival rate of the working group 1 is close to that of the Natural Chinese caterpillar fungus group.

Therapeutic Effects on the Kidney Replantation in Working Group 1-
Contrasting Group 1 and Natural Chinese Caterpillar Fungus Group

| | | Number of | Number of | |
|---|---|---|---|---|
| | Number of | Rats That | Rats That | Survival Rate |
| Group | Rats | died | Survived | (%) |
| Contrasting | 30 | 18 | 12 | 40% |
| Group 1 | | | | |
| Working | 30 | 2 | 28 | 93.3% |
| Group 1 | | | | |
| Natural Chinese | 30 | 1 | 29 | 96.7% |
| Caterpillar Fungus | | | | |
| Group | | | | |

Note:
For comparison between working group 1 and contrasting group 1: Probability value < 0.05; for comparison between working group 1 and natural Chinese caterpillar fungus group: Probability value > 0.05.

### Working Example 2

We have isolated new fungal strains in the growing area every year and then cultivated them at 16° C. through the sexual and asexual reproductive portions of their developmental cycle, during which stromata and conidia develop. Then the obtained fungal strains are introduced into 500 ml Erlenmeyer flasks for cultivation at 5° C., in order to identify whether the cultivated fungal strains are true Chinese caterpillar fungi. I.e., if any one fungal strain can develop stromata, it can be identified as a true Chinese caterpillar fungus, and if it cannot, it should be eliminated. The fungal strains, which have been utilized in production and have been purified and reproduced for more than 10 generation, must be rejuvenated in a high-nutritional solid culture medium at 5° C. for more than 5 generations. If as a result of comparison the conidia and conidiophores grown from rejuvenated fungal strains are identical to those from the fungal strains that are newly isolated from the growing area, can develop stromata in the Erlenmeyer flasks and can grow in colonies, it has been proved that there occurs no variation or very little variation, so the fugal strains can be still utilized in production

The formulation of the culture medium for the above-mentioned cultivation (calculated as 1000 g) comprises beef tea 400 g, lactalbumin hydrolysate 40 g, yeast powder 2 g, glucose 30 g, milk 150 g, nucleic acid 0.7 g, magnesium sulfate 0.3 g, sodium dihydrogen phosphate 0.08 g, compound vitamin 1 g, agar powder 18 g and water 500 g; the pH value of the culture medium is 7.6.

The fungal strains cultivated via said procedure are put into the culture medium that is put on a rocking device with the temperature of 16° C., and fermented at 16° C. for 10 days. After that the culture is expanded by 10 times and fermented stepwise. Until the culture medium is put into the production vessel of 20 to 30 tons at step 4, the fermentation is conducted in the liquid culture medium. The liquid culture medium (calculated from weight percent) comprises royal jelly 2 g, oat powder 20 g, milk powder 15 g, magnesium sulfate 0.3 g, dipotassium hydrogen phosphate and 0.6 g, vitamin 1 g in every 1000 g water; the pH value of said liquid culture medium is 7.2.

After that, the liquid culture medium containing hyphae is introduced into a drum vacuum dryer, and thereupon the hyphae are adsorbed at the bottom of the filter and slowly transferred via a belt conveyor through a 18-meter long constant-temperature drying tunnel at 60° C. while the hyphae are heated uniformly and oxidized consistently. When the culture medium containing the hyphae reaches the outlet, it has already lost 70% water. Subsequently it is cut into flakes of approx. 1.0×1.0 cm. Finally these flakes are quickly dried in a boiling dryer, pulverized and sifted to obtain the finished products.

### Working Example 3

We have isolated new fungal strains in the growing area every year and then cultivated them at 20° C. through the sexual and asexual reproductive portions of their developmental cycle, during which stromata and conidia develop. Then the obtained fungal strains are introduced into 500 ml Erlenmeyer flasks for cultivation at 10° C., in order to identify whether the cultivated fungal strains are true Chinese caterpillar fungi. I.e., if any one fungal strain can develop stromata, it can be identified as a true Chinese caterpillar fungus, and if it cannot, it should be eliminated. The fungal strains, which have been utilized in production and have been purified and reproduced for more than 10 generation, must be rejuvenated in a high-nutritional solid culture medium at 10° C. for more than 5 generations. If as a result of comparison the conidia and conidiophores grown from rejuvenated fungal strains are identical to those from the fungal strains that are newly isolated from the growing area and can develop stromata in colonies in the Erlenmeyer flasks, it has been proved that there occurs no variation or very little variation, so the fugal strains can be still utilized in production

The formulation of the culture medium for the above-mentioned cultivation (calculated as 1000 g) comprises beef tea 500 g, lactalbumin hydrolysate 5 g, yeast powder 10 g, glucose 20 g, milk 100 g, nucleic acid 1 g, magnesium sulfate 0.4 g, sodium dihydrogen phosphate 0.6 g, compound vitamin 0.5 g, agar powder 20 g and water 600 g; the pH value of the culture medium is 8.

The fungal strains cultivated via said procedure are put into the culture medium that is put on a rocking device with the temperature of 18° C., and fermented at 18° C. for 12 days. After that the culture is expanded by 8 times and fermented stepwise. Until the culture medium is put into the production vessel of 20 to 30 tons at step 4, the fermentation is conducted in the liquid culture medium. The liquid culture medium (calculated from weight percent) comprises silkworm chrysalis powder 15 g, protein peptone 2 g, corn flour 10 g, wheat gluten 15 g, lactalbumin hydrolysate 2 g, glucose 20 g, magnesium sulfate 0.3 g and dipotassium hydrogen phosphate 0.6 g in every 1000 g water; the pH value of said liquid culture medium is 7.5.

After that, the liquid culture medium (containing hyphae) is introduced into a drum vacuum dryer, and thereupon the hyphae are adsorbed at the bottom of the filter and slowly transferred via a belt conveyor through a 20 m long constant-temperature drying tunnel at 60° C. while the hyphae are heated uniformly and oxidized consistently. When the culture medium containing the hyphae reaches the outlet, it has already lost 70% water. Subsequently it is cut into flakes of approx. 1.2×1.2 cm. Finally these flakes are quickly dried in a boiling dryer, pulverized and sifted to obtain the finished products.

The finished product could also be obtained this way: the culture medium containing the hyphae is filtered via a drum vacuum filter and the resulting waste filtrate is filtered with a membrane again to eliminate some low-molecular materials. Then the filtrate undergoes spray drying and the resulting product is added to the fungal powder and the mixture is stirred uniformly.

According to a recent experiment, the Chinese caterpillar fungus, which has undergone the fermentation of 4 steps with the hyphae grown well, can be directly homogenized and then filtered through a membrane to eliminate part of low-molecular materials. Subsequently the filtrate is dried via spray drying to obtain the artificial Chinese caterpillar fungus powder. By using this process, the quantity of fungal derivants in the liquid culture medium is increased, and therefore the effectiveness is higher and the ingredients are more similar to those of natural Chinese caterpillar fungus.

The purpose of concrete working examples described in the invention is to illustrate the principles of the invention. The technical professionals, who work in the technical field related to the invention, can modified and supplement the described working examples in various ways, or replace them with similar methods, but the principles of the invention should not be deviated, and the range of the Claims attached should not be transgress.

What is claimed is:

1. An industrial fermentation-producing method for the asexual Chinese caterpillar fungus (Hirsutella hepiali Chen & Shen) comprises following steps:
1) performing an isolation of fungal strains, wherein the isolation step includes isolating new fungal strains in a growing area, cultivating the new fungal stains at 10-20° C., and then transferring fungal strains obtained into flasks for continuous cultivation;
2) performing a testing step, wherein the testing step includes cultivating the fungal strains that have been transferred into the flasks at 0-10° C., checking whether cultivated fungal strains can develop stromata, and selecting fungal strains that can develop stromata for production;
3) performing a rejuvenating cultivation, wherein the rejuvenating cultivation step includes purifying and reproducing the fungal strains selected for the production in step 2, rejuvenating fungal strains that have been purified and reproduced for more than 10 generations via cultivation in a solid culture medium, wherein the fungal strains are reproduced at a low temperature of 0-10° C. for more than 5 generations, and comparing and confirming that conidia and conidiophores that have grown via rejuvenating cultivation are identical to conidia and conidiophores of fungi that have been isolated from the growing area and that can develop stromata, the solid culture medium having a formulation, calculated as 1000 g, comprising beef tea (1:2) 300-500 g, lactalbumin hydrolysate 5-10 g, yeast powder 1 g, glucose 20-40 g, milk 100-200 g, nucleic acid 0.5-1 g, magnesium sulfate 0.1 g-0.4 g, potassium dihydrogen phosphate 0.6-1 g, compound vitamin 0.5-2 g, agar powder 15-20 g, and water 300-600 g, wherein a pH value of said medium is 7-8.
4) performing a second cultivation, wherein the second cultivation step includes introducing fungal strains from step 3, which have identical conidia and conidiophores into a liquid culture medium with a temperature of 12-20° C. for cultivation, and putting said liquid culture medium on a rocking device for cultivation of 6-12 days;
5) performing a fermentation step, wherein the fermentation step includes putting cultivated fungal strains from step 4 into a step-1 starter vat, fermenting at 12-20° C. in the liquid culture medium in step 4 for 8-12 days, expanding the liquid culture medium by 8-12 times and fermenting gradually, and then removing fermented fungal strains from the vat and drying them, wherein said liquid culture medium, calculated from weight percent, comprises at least one of following materials: carbon source 0.5-5 wt %, nitrogen source 0.5-2 wt %, trace elements 0.1-0.2 wt %, vitamins 0.1-0.2 wt %, or water.

2. The industrial fermentation-producing method of the asexual Chinese caterpillar fungus (Hirsutella hepiali Chen & Shen) according to claim 1, wherein the rejuvenating cultivation step comprises cultivating with the solid culture medium that, calculated from weight percent, comprises: beef tea (1:2) 300-500 g, lactalbumin hydrolysate 5-10 g, yeast powder 1 g, glucose 20-40 g, milk 100-200 g, nucleic acid 0.5 g, magnesium sulfate 0.2 g, potassium dihydrogen phosphate 1 g, the compound vitamin 1 g, agar powder 15 g and water 400-500 g, a pH value of said culture medium being 7.2-7.6.

3. The industrial fermentation-producing method of the asexual Chinese caterpillar fungus (Hirsutella hepiali Chen & Shen) according to claim 1, wherein the nitrogen source for the liquid culture medium, calculated from weight percent, comprises at least one of following materials: silkworm chrysalis powder, protein peptone, milk powder, yeast powder or lactalbumin hydrolysate, the carbon source for the liquid culture medium, calculated from weight percent, comprising at least one of following materials: royal jelly, oat powder, wheat gluten, saccharose, corn flour or glucose, the trace elements for the liquid culture medium, calculated from weight percent, comprising at least one of following materials: magnesium sulfate, dipotassium hydrogen phosphate, or rare earth elements.

4. The industrial fermentation-producing method of the asexual Chinese caterpillar fungus (Hirsutella hepiali Chen & Shen) according to claim 3, wherein for every 1000 g water, the liquid culture medium comprises silkworm chrysalis powder 15 g, protein peptone 2 g, corn flour 10 g, wheat gluten 15 g, glucose 20 g, magnesium sulfate 0.3 g, and dipotassium hydrogen phosphate 0.6 g, a pH value of said liquid culture medium being 7.0-7.5.

5. The industrial fermentation-producing method of the asexual Chinese caterpillar fungus (Hirsutella hepiali Chen & Shen) according to claim 3, wherein, for every 1000 g water, the liquid culture medium comprises royal jelly 2 g, oat powder 20 g, milk powder 15 g, saccharose 20 g, magnesium sulfate 0.3 g, dipotassium hydrogen phosphate 0.6 g, rare earth elements 1 g, and vitamin 1 g, a pH value of said liquid culture medium being 7.0-7.5.

6. The industrial fermentation-producing method of the asexual Chinese caterpillar fungus (Hirsutella hepiali Chen & Shen) according to claim 1, wherein the compound vitamin is prepared by blending vitamin B1, vitamin B2, thiamin and riboflavin with water.

7. The industrial fermentation-producing method of the asexual Chinese caterpillar fungus (Hirsutella hepiali Chen & Shen) according to claim 1, further comprising introducing the liquid culture medium containing hyphae from step 5 into a high-level storing vessel, slowly transferring the hyphae into a drum vacuum dryer, where the hyphae are evenly adsorbed at the bottom, then gradually sending the hyphae through a 15-20 m long drying tunnel, where the culture medium containing the hyphae has already lost 70-80% water when reaching the outlet of the drying tunnel, cutting the hyphae into flakes of approximately 1.0×1.0 cm with a cutter, and boiling, drying, pulverizing and sifting the flakes to obtain finished products.
